(19) United States
(12) Patent Application Publication
(10) Pub. No.: US 2011/0021421 A1
(43) Pub. Date: Jan. 27, 2011

(54) LIQUID ENTERAL NUTRITIONAL COMPOSITION WITH A LOW SPECIFIC PROTEIN VOLUME

(75) Inventors: Wynette Hermina Agnes Kiers, Heteren (NL); Marcel Minor, Wageningen (NL); Thomas Hubertus Martinus Snoeren, Steenwijk (NL); Edward Lucian Sliwinski, Oss (NL)

Correspondence Address: FOLEY AND LARDNER LLP;SUITE 500, 3000 K STREET NW, WASHINGTON, DC, 20007, US

(73) Assignee: N.V. NUTRICIA

(21) Appl. No.: 12/746,135

(22) PCT Filed: Dec. 5, 2008
(86) PCT No.: PCT/NL08/50776

§ 371 Date: Sep. 16, 2010

Related U.S. Application Data
(60) Provisional application No. 61/059,865, filed on Jun. 9, 2008.

(30) Foreign Application Priority Data
Dec. 5, 2007 (NL) PCT/NL2007/050626
Mar. 12, 2008 (NL) PCT/NL2008/050141
Jun. 9, 2008 (EP) 08157877.5
Nov. 14, 2008 (EP) 08169152.9

(51) Int. Cl. A23L 1/305; A61K 38/17; A61P 3/02; A61P 3/00; A23J 3/08; A23J 3/14; A23L 1/29

(52) U.S. Cl. 514/5.6; 514/5.5; 426/580; 426/590; 426/474; 426/2

(57) ABSTRACT

The present invention concerns liquid enteral nutritional compositions that comprise intact protein with a low specific volume allowing the preparation of nutrition with high protein and energy contents which are particularly advantageous for persons that are in, or recovering from, a disease state, persons that are malnourished or sportsmen and women and active elderly.

### FIELD OF THE INVENTION

The present invention is in the field of liquid enteral nutritional compositions.

### BACKGROUND OF THE INVENTION

The present invention relates in general to a shelf-stable liquid enteral composition for providing nutrition, either as a supplement, or as a complete nutrition, comprising an intact protein having a low specific volume in solution. Preferably, said intact protein is a plant or a milk protein.

Some patients need nutrition, either as a supplement, or as a complete nutrition, in the smallest volume of liquid.

These patients can be cachectic patients or persons suffering from end-stage AIDS, cancer or cancer treatment, severe pulmonary diseases like COPD (chronic obstructive pulmonary disease), tuberculosis and other infection diseases or persons that experienced severe surgery or trauma like burns. Furthermore, persons suffering from disorders in the throat or mouth such as oesophageal cancer or stomatitis and persons having problems with swallowing like dysphagic persons, require special liquid, low-volume nutrition. Also, persons just suffering from reduced appetite or loss of taste, will benefit from low-volume, preferably liquid, food.

These patients can also be elderly persons, in particular frail elderly and elderly at risk of becoming frail. In this regard, although an elderly person's energy needs may be reduced, their ability to consume products may also be diminished. For example, they may have difficulty consuming a product due to, e.g., swallowing difficulties, or due the too large amount of product they need to consume to meet the daily intake of nutrients. Hence, compliance is not optimal, and often, the intake is suboptimal, leading to suboptimal nourishment, and in the end, to malnutrition.

In this respect, it is submitted that in the context of this application, an elderly person is a person of the age of 50 or more, in particular of the age of 55 or more, more in particular of the age of 60 or more, more in particular of the age of 65 or more. This rather broad definition takes into account the fact that the average age varies between different populations, on different continents, etc. Most developed world countries have accepted the chronological age of 65 years as a definition of ‘elderly’ or older person (associated with the age at which one may begin to receive pension benefits), but like many westernized concepts, this does not adapt well to e.g. the situation in Africa. At the moment, there is no United Nations (UN) standard numerical criterion, but the UN agreed cut-off is 60+ years to refer to the older population in Western world. The more traditional African definitions of an elder or ‘elderly’ person correlate with the chronological ages of 50 to 65 years, depending on the setting, the region and the country.

The aforementioned groups of patients may be extremely sensitive to food consistency and to the organoleptic properties of the product such as, for instance viscosity, mouth feel, taste, smell and colour. Also, patients such as cachectic patients, typically suffer from extreme weakness which often prevents them from sitting in a vertical position and from drinking food from a carton or even to suck it from a straw. These patients benefit well from liquid low-volume enteral compositions with a high content of nutrients, in particular proteins.

However, increasing calories and/or proteins in a nutritional liquid composition may increase the overall viscosity of the composition. This can make the liquid nutritional composition difficult to consume or administer, and can also diminish the taste of the nutritional composition. Furthermore, technical difficulties exist in producing a stable, in particular a shelf-stable nutritional liquid composition having a high content of proteins.

Therefore, the problem underlying the present invention is to provide a shelf-stable liquid enteral composition for providing nutrition, either as a supplement, or as a complete nutrition, comprising an intact protein having a low specific volume in solution, as major protein source, in the smallest volume of liquid, and which supports nutrition and well-being in the different patient groups mentioned above, in particular to an elderly person or an ill patient.

Major technical difficulties exist in producing such a shelf-stable liquid enteral nutritional composition with a high content of proteins, in particular intact proteins.

For example, in comparison to common milk, increasing the amount of proteins leads to precipitation and sedimentation of proteins and other ingredients, such as lipids and digestible carbohydrates, which imparts nutrient intake.

Concentrating liquids also increases the chance of undesired interactions between ingredients which reduces stability, especially during heating and long-term storage. Shelf-stable is defined as having a stability of more than 6 months on the shelf under normal storage conditions, i.e. at an ambient temperature of between 18 and 25° C., and at a standard atmospheric pressure.

Furthermore, increasing the protein content in a nutritional liquid composition may increase the overall viscosity of the composition. This can make the liquid nutritional composition difficult to consume or administer, and can also diminish the taste of the nutritional composition. These phenomena often follow non-linear kinetics and the problems quickly increase in magnitude when the concentration of ingredients is increased above 28 weight %. Therefore, many of the commercial shelf-stable liquid products that are currently available have intact protein levels below about 9 g per 100 ml of product.

A known solution to the problem how to increase the protein content to a higher level without imparting viscosity is replacing part of the total protein by peptides or free amino acids. However, this seriously decreases taste appreciation and therefore voluntary intake of the nutritional composition by the patient group.

On the other hand, many concentrates like condensed milks suffer from an incomplete nutrient profile, too high lactose levels, sticky mouth-feel, high viscosity, extreme sweetness and a high osmotic value, which is not appreciated by the consumer and increases rapidly feelings of fullness and satiety after consumption. This makes that the urge to consume more volume deteriorates rapidly once a small amount of the product has been consumed.

### PRIOR ART

WO 02/098242 A1 (Nestlé, 12 Dec. 2002) discloses a calorically dense liquid oral supplement (2.25 kcal/ml) based on a (60:40) soy protein isolate/caseinate mixture with a protein level of 9 g/100 ml (16 En %), 12.25 g/100 ml of fat (49 En %), and 19.7 g/100 ml of digestible carbohydrates (35 En %).

The commercially available product RESOURCE® 2.0 is a high calorie product from Novartis (2 kcal/ml), based on a mixture of calcium and sodium caseinate as protein source, comprising 9 g/100 ml of proteins (18 En %), 8.7 g/100 ml of fat (39 En %), and 21.4 g/100 ml of digestible carbohydrates (43 En %), and is provided in a 237 ml unit dosage.

The commercially available product VHC® 2.25 is a high calorie product from Nestlé (2.25 kcal/ml), based on a mixture of calcium- and potassium caseinate and isolated soy protein as protein source, comprising 9 g/100 ml of proteins (16 En %), 12 g/100 ml of fat (48 En %) and 19.7 g/100 ml of digestible carbohydrates (35 En %), and is provided in a 250 ml unit dosage.

The commercially available product FRESUBIN® 2.0 is a high calorie product from Fresenius (2 kcal/ml), based on milk proteins as protein source, comprising 10 g/100 ml of proteins (20 En %), 7.8 g/100 ml of fat (35 En %), and 22.5 g/100 ml of digestible carbohydrates (45 En %), and is provided in a 200 ml unit dosage.

The commercially available product PRO-CAL SHOT® is a high calorie product from Vitaflo International Ltd (3.34 kcal/ml), based on skimmed milk powder and sodium caseinate as protein source, comprising 6.7 g/100 ml of proteins (8 En %), 28.2 g/100 ml of fat (76 En %), and 13.4 g/100 ml of digestible carbohydrates (16 en %), and is provided in a 250 ml unit dosage.

The commercially available product TwoCal® HN is a high calorie product from Abbott Laboratories (Ross Nutrition) (2 kcal/ml), based on sodium and calcium caseinate as protein source, comprising 8.4 g/100 ml of proteins (16.7 En %), 8.9 g/100 ml of fat (40.1 En %), and 21.6 g/100 ml of digestible carbohydrates (43.2 En %), and is provided in a 237 ml unit dosage.

Ferreira et al., Journal of Food Science, 60 (1): 92, 1995 disclose the specific volume of a number of protein foams (whipped protein solutions) made from solutions of different proteins. The concept of specific volume, as used in said publication, refers to the volume/weight ratio of the solution per se after whipping, and is not related to the specific volume as defined in this invention, being the volume occupied by a unit of protein in a nutritional composition according to the invention.

Monkos, Journal of Biological Physics 31: 219-232, 2005 discloses a number of hydrated human proteins (IgG, Human Serum Albumin (HSA), ovalbumin and lysozyme) and Bovine Serum Albumin (BSA) and their specific volume in a solution. Such proteins are clearly not intended for, nor usable as a source of proteins suitable for preparing an enteral nutritional composition, nor are the specific volume values cited for these proteins obtained after a heat-treatment (sterilisation or pasteurisation), contrary to the specific volume values in our invention.

### SUMMARY OF THE INVENTION

The present invention provides a liquid enteral nutritional composition comprising an intact protein having a low specific volume in solution, designed to meet the nutritional needs of persons in need thereof, in particular elderly and patients with certain disease states. The composition provides an increased amount of energy per unit volume while providing a sufficiently low viscosity to allow the composition to be easily consumed orally or be administered by tube. In addition, the taste of the composition is not deteriorated.

To this end, in a first aspect of the present invention, a liquid enteral nutritional composition is provided, comprising an intact protein having a specific volume in said composition of less than 3.30 ml/g.

More preferably, a liquid enteral nutritional composition is provided, comprising an intact protein having a specific volume in said composition of less than 3.25 ml/g.

Still more preferably, a liquid enteral nutritional composition is provided, comprising an intact protein having a specific volume in said composition of less than 3.20 ml/g. According to further embodiments, a liquid enteral nutritional composition is provided, comprising an intact protein having a specific volume in said composition of less than 3.15 ml/g, less than 3.10 ml/g, less than 3.00 ml/g, less than 2.95 ml/g, less than 2.90 ml/g, less than 2.85 ml/g, less than 2.80 ml/g, less than 2.75 ml/g, less than 2.70 ml/g, less than 2.65 ml/g, less than 2.60 ml/g, less than 2.55 ml/g, less than 2.50 ml/g, less than 2.45 ml/g, less than 2.40 ml/g, less than 2.35 ml/g, less than 2.30 ml/g, less than 2.25 ml/g, less than 2.20 ml/g, less than 2.15 ml/g, less than 2.10 ml/g, less than 2.05 ml/g less or than 2.00 ml/g.

In one embodiment according to the present invention, the intact protein is preferably a milk protein.

In a further aspect, the present invention concerns a method of providing nutrition to a person in need thereof, comprising the steps of administering to said person the nutritional composition according to the present invention.

In the context of this invention, the term “at least” also includes the starting point of the open range. For example, an amount of “at least 95 weight %” means any amount equal to 95 weight % or above.

In the context of this invention, enteral means orally or by tube.

In the context of this invention, the % of total energy is also abbreviated as En %; En % is thus short for energy percentage and represents the relative amount that a constituent contributes to the total caloric value of the composition.

In the context of this invention, the term “about” indicates that a certain deviation is allowed from a cited value, the magnitude thereof being determined by inter alia the accuracy of the determination method. Typically, such a deviation is 10%.

In the context of this invention, “non-hydrolysed” proteins is equivalent to “intact” proteins, meaning that the proteins have not, or not substantially, been subjected to a hydrolysis process. However, minor amounts of hydrolysed proteins may be present in the source of non-hydrolysed proteins, or may be added to the formulation, such as additional amino acids, such as for example branched chain amino acids, for example leucine, isoleucine, valine and the like. In this context, “minor” should be understood as an amount of about 10 weight % or less, based on total protein.

The invention will now be further elucidated by describing the preferred embodiments of the present invention in more detail.

### DETAILED DESCRIPTION OF THE INVENTION

The volume fraction of a dairy, or dairy-like system based nutritional composition comprising an intact protein and optionally one of digestible carbohydrate, dietary fiber and fat can be described by formula (f1)

φₙ(Cₚ·vₚ+C_{c}·v_{c}+C_{d}·v_{d}+C_{f}·v_{f} (f1)

wherein
the indices n, p, c, d and f stand for nutritional composition (n), intact protein (p), digestible carbohydrate (c), dietary fiber (d) and fat (f), respectively;
φ is the volume fraction (dimensionless);
C is the absolute concentration of a material; and
v is the specific volume, i.e. the volume occupied by a unit of mass of a material;
which can be calculated for each dairy system using literature values, or it can be measured, e.g. viscometrically (see e.g. Journal of Dairy Science Vol. 56, No. 6, 1972). It is known in the art that the maximum volume fraction φₘₐₓ for dairy systems, comprising protein, digestible carbohydrate and fat is about 0.79. In the context of the present invention, it is assumed that only protein, digestible carbohydrate, dietary fiber and fat contribute to the volume fraction.

Moreover, using Eilers' equation (f2) (Eilers, H. 1941, Kolloid Z. 97:313, Eilers, H. 1942, Kolloid Z. 102:154 and Snoeren et al. NIZO-nieuws 1983, nr. 1),

ηₙ=η_{solvent{}1+1.25φₙ/[φₘₐₓ−φₙ)/φₘₐₓ]}² (f2)

which shows the relationship between the viscosity of a nutritional composition ηₙ (usually expressed in mPa·s) and the volume fraction φₙ of a dairy system, wherein ηₛₒₗᵥₑₙₜ is 1 mPa·s, being the viscosity of water and φₘₐₓ is 0.79, it can be shown that for a volume fraction of about 0.73, the viscosity of said dairy system becomes so high that the dairy system is not a liquid as defined herein anymore. A value of about 200 mPa·s is herewith defined as an empirical upper viscosity limit, above which a liquid system has an unacceptably high viscosity to be readily drinkable. In the context of this invention, it is understood that “liquid” refers to a water-based composition, such as a solution or a suspension, having a viscosity of 200 mPa·s or less, as determined at 20° C. in a rotational rheometer at a shear rate of 100 s⁻¹.

It was an object of the present invention to provide a stable, attractive, easily drinkable, liquid enteral composition for providing nutrition, either as a supplement, or as a complete nutrition, with a high energy content, to a person, in particular to an elderly person or an ill patient.

To this end, the inventors have found that such a composition could be provided if the specific volume of the protein (vₚ) was decreased. As a consequence, it is possible to introduce more protein in a nutritional dairy-based composition, yielding—at the same viscosity—a higher protein concentration, protein energy content and a higher total energy content.

Consequently, the present invention provides a liquid enteral nutritional composition, comprising an intact protein having a specific volume in said composition of less than 3.30 ml/g. Currently known prior art specific volumes for intact protein in an enteral nutritional formulation are equal to or higher than 3.30 ml/g.

### Determination of Specific Volume.

In the context of this invention, the specific volume of the intact protein in a liquid enteral nutritional composition, optionally comprising one or more of digestible carbohydrate, dietary fiber and fat, is calculated on the basis of the viscosity of the liquid nutritional product using equations (f1) and (f2), in which first the volume fraction is calculated using (f2), and next the specific volume of the protein using (f1). To this end, the viscosity of the product is measured as further described here below. This method is preferred because it enables determination of the specific volume of intact protein leaving the total composition of a complex liquid nutritional product unchanged.

Furthermore, the energy content of the liquid enteral nutritional composition may also be calculated using formula (f3):

Enₙ=Cₚ·Eₚ+C_{c}·E_{c}+C_{f}·E_{f} (f3)

wherein
the indices n, p, c and f stand for nutritional composition (n), intact protein (p), digestible carbohydrate (c), and fat (f), respectively;
C is the absolute concentration of a material;
En is the energy content of the liquid enteral nutritional composition; and
E is the energy value of a material.

According to this model description, it is implied that the contribution of the dietary fiber to the total energy of the composition is minimal, and may be ignored as in formula (f3). However, the effects of the dietary fiber on the viscosity, and hence on the volume fraction, may not be ignored, and are included in the formula (f1).

In practice, the following values are applicable: Eₚ=4 kcal/g, E_{c}=4 kcal/g; E_{f}=9 kcal/g; v_{c}=1.05 ml/g; v_{d}=1.05 ml/g and v_{f}=1.12 ml/g. These values are taken from Nutricia Vademecum, Elsevier, Maarssen, 1998. A common prior art value for vₚ=3.30 ml/g.

Hence, the formulas (f1) and (f3) reduce to

0.73=Cₚ·3.30+C_{c}·1.05+C_{d}·1.05+C_{f}·1.12 (f4)

Enₙ=Cₚ·4+C_{c}·4+C_{f}·9 (f5)

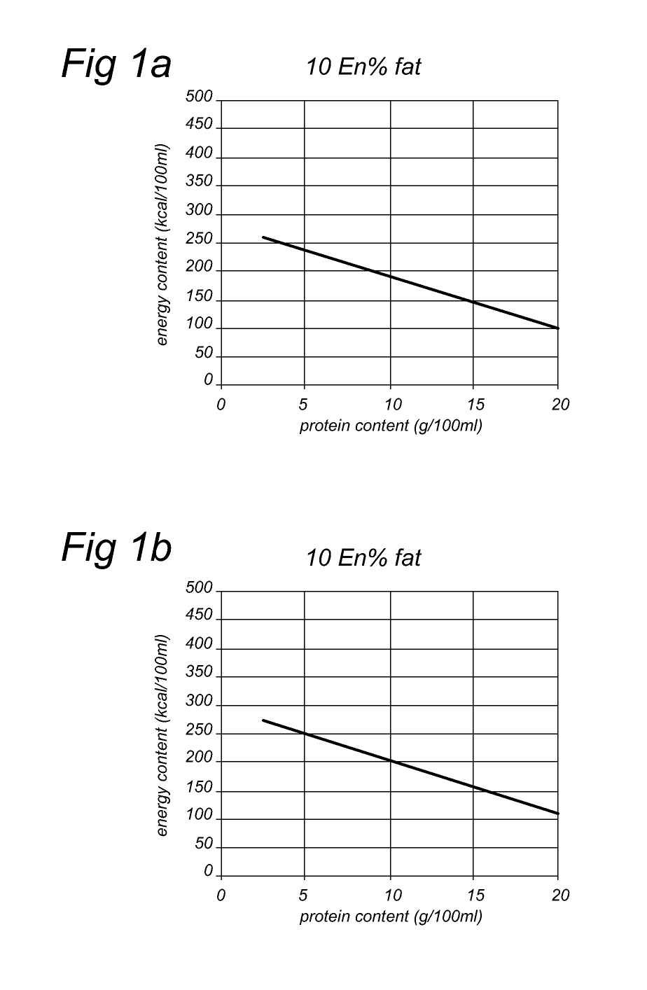

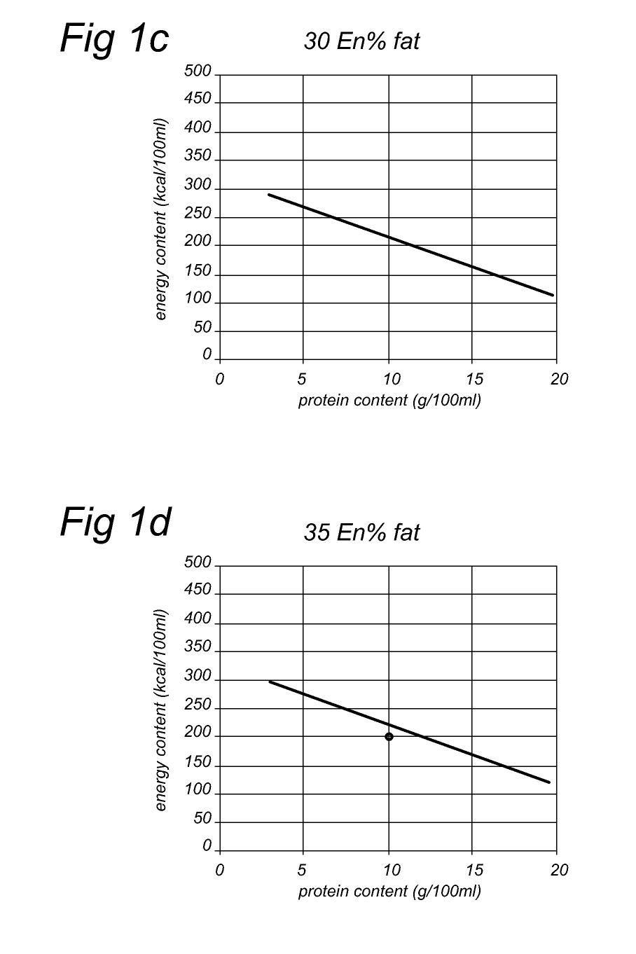

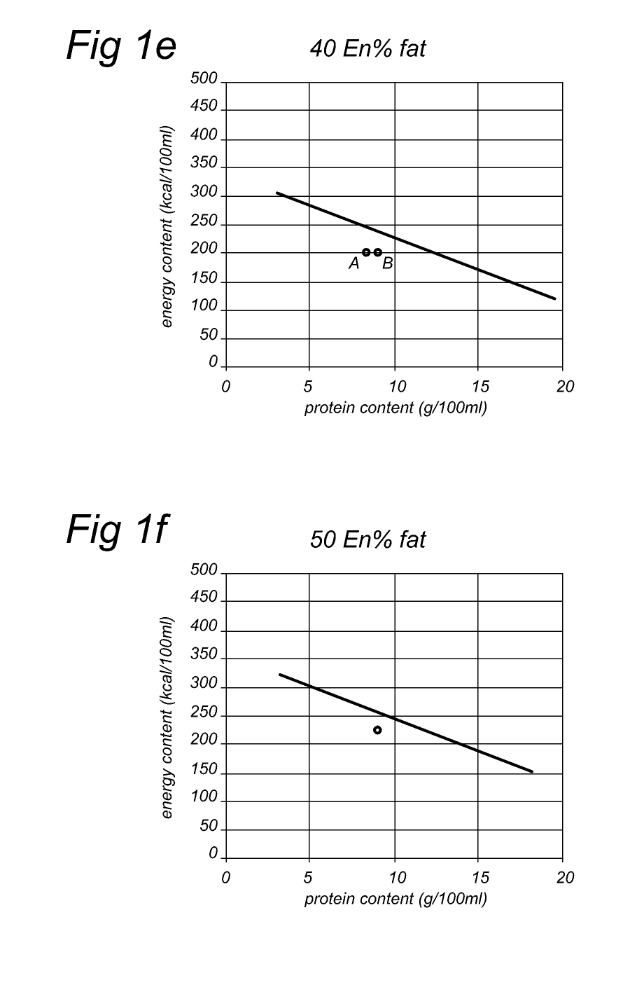

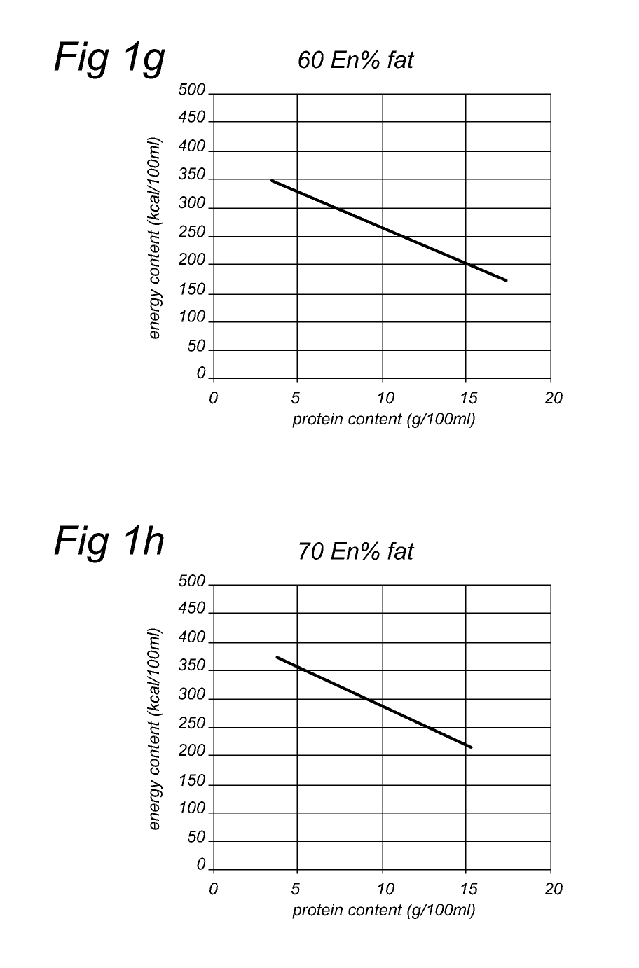

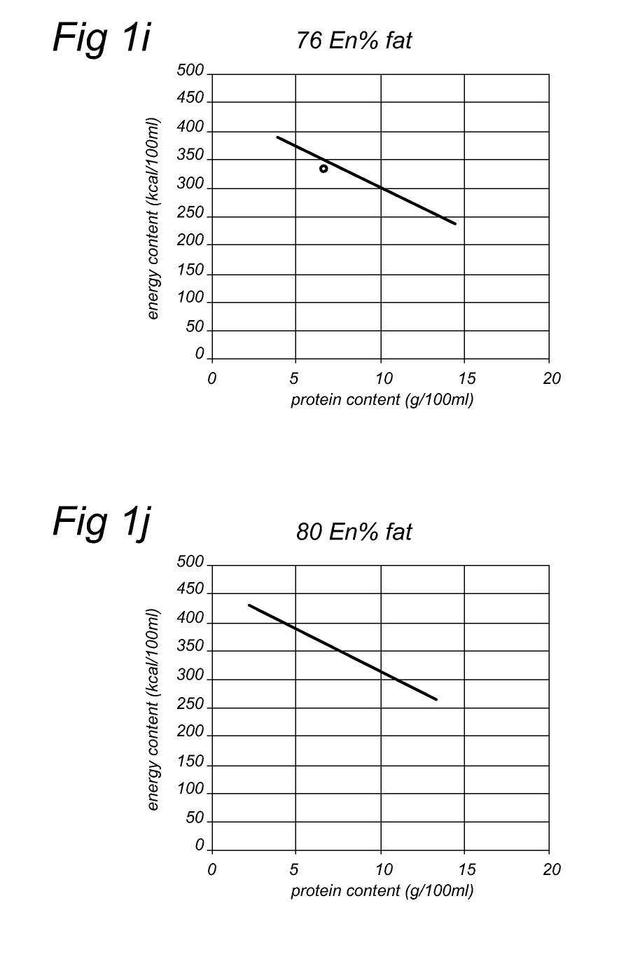

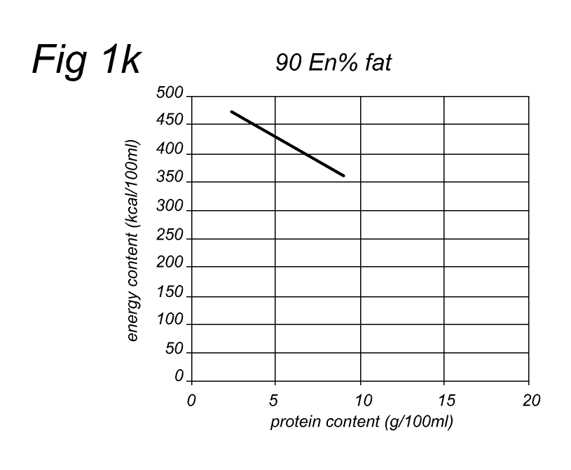

and can be solved for Enₙ and Cₚ defining compositions for each concentration of fat C_{f}. A set of solutions is shown in FIGS. 1a-k between certain (practical) values for Cₚ. For example, for a composition comprising 30 En % of fat, the present invention provides compositions for which the value of energy content and protein content are situated above said curve, i.e. compositions which comprise intact protein with a specific volume of less than 3.30 ml/g. In the exemplified solutions, the amount of dietary fiber is taken to be zero. In order to make the volume fraction φ dimensionless, the concentration C in (f4) is expressed as g/ml.

In a further aspect of the invention, a liquid enteral nutritional composition according to the invention is provided, comprising an intact protein and optionally one or more of digestible carbohydrate, dietary fiber and fat, wherein the relationship between the intact protein, digestible carbohydrate, dietary fiber and fat and the volume fraction is described by formula (f6):

[Cₚ·v_{p′}+C_{c}·1.05+C_{d}·1.05+C_{f}·1.12]/φₙ>1 (f6)

wherein φₙ is defined as in formula (f1) and can be calculated using experimentally determined values and v_{p′} is equal to 3.30 ml/g, the common prior art value for vₚ. As far as we know, the prior art does not disclose any liquid enteral nutritional composition with a vₚ-value lower than 3.30 ml/g. However, in the event the prior art should disclose a value for vₚ lower than 3.30 ml/g, in a further aspect of the invention, a liquid enteral nutritional composition according to the invention is provided, comprising an intact protein and optionally one or more of digestible carbohydrate, dietary fiber and fat, wherein the relationship between the intact protein, digestible carbohydrate, dietary fiber and fat and the volume fraction is described by formula (f6) and wherein v_{p′} is the lowest value cited in the prior art, such as, for instance 3.25 ml/g, 3.20 ml/g, 3.15 ml/g, 3.10 ml/g, 3.00 ml/g, 2.95 ml/g, 2.90 ml/g, 2.85 ml/g, 2.80 ml/g, 2.75 ml/g, 2.70 ml/g, 2.65 ml/g, 2.60 ml/g, 2.55 ml/g, 2.50 ml/g, 2.45 ml/g, 2.40 ml/g, 2.35 ml/g, 2.30 ml/g, 2.25 ml/g, 2.20 ml/g, 2.15 ml/g, 2.10 ml/g, 2.05 ml/g or 2.00 ml/g.

### Intact Protein

Preferably, the intact proteins comprise or consist of intact milk proteins. According to the invention, intact milk protein is defined as a milk protein in its native state originating from milk. According to one embodiment of the present invention, said intact protein comprises micellar casein. According to another embodiment of the present invention, said intact protein comprises whey proteins. It is understood that, when referring to intact milk proteins, substantially all milk proteins are intact. In further embodiments the intact milk protein comprises milk protein concentrate (MPC) and/or milk protein isolate (MPI).

Although a composition comprising the intact proteins according to the invention is preferably a composition with a high amount of proteins, preferably in a small volume, there is no restriction to the amount of proteins that may be present in the composition. Amounts may vary from very low amounts as low as 1 g/100 ml, or less to very high amounts, as high as 20 g/100 ml, or more, depending on e.g. the required energy content of the composition.

The intact protein may also be a protein of plant origin, such as pea protein or soy protein.

### Nutritional Composition

The composition according to the invention is designed to either supplement a person's diet or to provide complete nutritional support. Hence, the composition according to the invention may further comprise at least one of the following components: fat, digestible carbohydrates, dietary fibers, vitamins, minerals and the like. Preferably, the composition according the invention is a nutritionally complete composition.

### Fat

In one embodiment the present liquid enteral nutritional composition further comprises fat. The amount of fat may range between 5 and 95 En %, preferably between 10-70 En %, more preferably between 20 to 40 En %, relative to the total energy content of the composition.

With regard to the type of fat, a wide choice is possible, as long as the fat is of food quality.

The fat may either be an animal fat or a vegetable fat or both. Although animal fats such as lard or butter have essentially equal caloric and nutritional values and can be used interchangeably, vegetable oils are highly preferred in the practice of the present invention due to their readily availability, ease of formulation, absence of cholesterol and lower concentration of saturated fatty acids. In one embodiment, the present composition comprises rapeseed oil, corn oil and/or sunflower oil.

The fat may include a source of medium chain fatty acids, such as medium chain triglycerides (MCT, mainly 8 to 10 carbon atoms long), a source of long chain fatty acids, such as long chain triglycerides (LCT) and phospholipid-bound fatty acids such as phospholipid-bound EPA or DHA, or any combination of the two types of sources. MCTs are beneficial because they are easily absorbed and metabolized in a metabolically-stressed patient. Moreover, the use of MCTs will reduce the risk of nutrient malabsorption. LCT sources, such as canola oil, rapeseed oil, sunflower oil, soybean oil, olive oil, coconut oil, palm oil, linseed oil, marine oil or corn oil are beneficial because it is known that LCTs may modulate the immune response in the human body.

In one specific embodiment, the fat comprises 30 to 60 weight % of animal, algal or fungal fat, 40 to 70 weight % of vegetable fat and optionally 0 to 20 weight % of MCTs based on total fat of the composition. The animal fat preferably comprises a low amount of milk fat, i.e. lower than 6 weight %, especially lower than 3 weight % based on total fat. In particular, a mixture of corn oil, egg oil, and/or canola oil and specific amounts of marine oil are used. Egg oils, fish oils and algal oils are a preferred source of non-vegetable fats. Especially for compositions that are to be consumed orally, in order to prevent formation of off-flavours and to decrease a fishy after-taste, it is recommended to select ingredients that are relatively low in docosahexanoic acid (DHA), i.e. less than 6 weight %, preferably less than 4 weight % based on total fat. Marine oils containing DHA are preferably present in the composition according to the invention in an amount lower than 25 weight %, preferably lower than 15 weight % based on total fat. On the other hand, inclusion of eicosapentanoic acid (EPA) is highly desirable for obtaining the maximum health effect. Therefore, in another embodiment, the amount of EPA may range between 4 weight % and 15 weight %, more preferably between 8 weight % and 13 weight % based on total fat. The weight ratio EPA:DHA is advantageously at least 6:4, for example between 2:1 and 10:1. In yet another embodiment, the amount of EPA is very low, such as 0.1 to 1 weight %, preferably 0.3 weight % or 0.6 weight %, based on total fat.

Also, the liquid nutritional composition according to the invention may beneficially comprise an emulsifier. Commonly known emulsifiers may be used and generally the emulsifier contributes to the energy content of the fat in said composition.

### Digestible Carbohydrate

In one embodiment, the present liquid enteral nutritional composition further comprises digestible carbohydrate. Preferably, said digestible carbohydrate provides between 30 to 60% of the total energy content of the composition. The digestible carbohydrate may comprise either simple or complex carbohydrates, or any mixture thereof. Suitable for use in the present invention are glucose, fructose, sucrose, lactose, trehalose, palatinose, corn syrup, malt, maltose, isomaltose, partially hydrolysed corn starch, maltodextrins, glucose oligo- and poly-saccharides. Preferably, the composition of the digestible carbohydrate is such that high viscosities, excessive sweetness, excessive browning (Maillard reactions) and excessive osmolarities are avoided. Acceptable viscosities and osmolarities may be achieved by adjusting the average chain length (average degree of polymerisation, DP) of the digestible carbohydrates between 1.5 and 6, preferably between 1.8 and 4. In order to avoid excessive sweetness, the total level of sucrose and fructose is less than 52% and preferably less than 40% of the weight of the digestible carbohydrate. Long-chain digestible carbohydrates such as starch, starch fractions and mild starch hydrolysates (DP≧6, DE<20), may also be present, preferably in an amount of less than 25 weight %, especially less than 15 weight % of the digestible carbohydrate, and less than 6 g/100 ml, preferably less than 4 g/100 ml of the liquid enteral composition according to the invention.

In one embodiment of the present invention, the digestible carbohydrate includes maltodextrose with a high DE (dextrose equivalent). In one embodiment the digestible carbohydrate includes maltodextrose with a DE of >20, preferably >30 or even >40, such as a DE of about 47. Surprisingly, the use of maltodextrose leads to few or no Maillard reaction products upon heating. Without being bound to any explanation, this effect might be attributed to the fact that the compact micellar structure of the micellar casein offers few lysine reaction sites for a Maillard reaction. In one embodiment of the present invention, the digestible carbohydrate includes maltodextrose with a high DE in an amount of at least 35 weight %, preferably at least 50 weight %, preferably at least 65 weight %, preferably at least 90 weight % of the digestible carbohydrate. In one embodiment of the present invention, the digestible carbohydrate includes maltodextrose with a low DE of 2 to 20. In one embodiment of the present invention, the digestible carbohydrate includes maltodextrose with a low DE of 2 to 10, preferably with a low DE of about 2. In one embodiment of the present invention, the digestible carbohydrate includes maltodextrose with a low DE in an amount of less than 35 weight %, preferably less than 20 weight %, preferably less than 10 weight % of the digestible carbohydrate. Maltodextrose with a low DE may also be referred to as maltodextrine. In another embodiment of the present invention, the digestible carbohydrate includes maltodextrose with a high DE, preferably a DE of >20, preferably >30 or even >40, most preferably a DE of about 47 in combination with maltodextrose with a low DE, preferably a low DE of 2 to 20, more preferably a low DE of 2 to 10, most preferably with a low DE of about 2. As is known, maltodextrose with a low DE, such as of about 2, gives rise to a high viscosity. Maltodextrose with a high DE, such as of about 47 gives rise to a low viscosity, but is very sweet. The combination of both maltodextroses optimizes the balance between sweetness and viscosity. In one embodiment of the present invention, the digestible carbohydrate includes at least 65 weight %, preferably at least 90 weight %, based on total digestible carbohydrate of maltodextrose with a DE>40, preferably with a DE of about 47 and 0 to 10 weight % of maltodextrose with a DE 2 to 10, preferably with a DE of about 2.

In another embodiment of the present invention, the digestible carbohydrate includes trehalose. As was indicated, it is one of the main objects of the invention to provide a nutritional composition with a low viscosity. Sucrose is very well suited for such purpose, but gives rise to very sweet compositions, which are in general disliked by the consumer. Trehalose is a preferred choice of digestible carbohydrate, as it gives rise to a low viscosity, no undesired Maillard reactions and it has a sweetness about half of that of sucrose. In one embodiment of the present invention, the digestible carbohydrate includes trehalose in an amount of 20% to 60% of the weight of the digestible carbohydrate, in an amount of 20% to 45%, more preferably in an amount of 25% to 45% of the weight of the digestible carbohydrate.

### Vitamins and Minerals

The compositions according to the invention may contain a variety of vitamins and minerals. Overall, the composition according to the invention preferably includes at least 100% of the United States Recommended Daily Allowance (USRDA) of vitamins and minerals in a one litre portion.

In one embodiment of the present invention, the composition according to the invention provides all necessary vitamins and minerals. For example, the composition according to the invention preferably provides 6 mg of zinc per 100 ml of the composition which is beneficial for tissue repair in a healing patient. Preferably, the composition according to the invention (also) provides 25 mg of vitamin C per 100 ml of the composition to aid patients with more severe healing requirements. Further, preferably, the composition according to the invention (also) provides 2.25 mg iron per 100 ml of the composition. Iron is beneficial in maintaining bodily fluids as well as circulatory system functions in an elderly patient.

In another embodiment of the present invention, the amount of divalent ions ranges between 170 mg/100 ml and 230 mg/100 ml and preferably between 180 mg/100 ml and 220 mg/100 ml. Preferably, the amount of calcium ranges between 155 mg/100 ml and 185 mg/100 ml and preferably between 160 mg/100 ml and 180 mg/100 ml. The phosphorus content can be above 10 mg per g of protein, with a calcium to phosphorus weight ratio between 1.0 and 2.0, preferably between 1.1 and 1.7. Carnitin may advantageously be present in an amount of 8 mg/100 ml to 1000 mg/100 ml, preferably 10 mg/100 ml to 100 mg/100 ml of composition; it may have the form of carnitin, alkyl carnitin, acyl carnation or mixtures thereof. Organic acids are preferably present at a level of between 0.1 g/100 ml to 0.6 g/100 ml, especially 0.25 g/100 ml to 0.5 g/100 ml. These acids include short fatty acids such as acetic acid, hydroxy acids such as lactic acid, gluconic acid, and preferably polyvalent hydroxy acids, such as malic acid and citric acid. In one embodiment of the present invention, the present composition also comprises citric acid.

### Non-Digestible Carbohydrates

The liquid enteral nutritional composition according to the invention may optionally be fortified with non-digestible carbohydrates (dietary fibres) such as fructo-oligosaccharides or inulin. In an embodiment of the present invention, the composition according to the invention comprises 0.5 g/100 ml to 6 g/100 ml of non-digestible carbohydrates. The dietary fibres include non-digestible oligosaccharides having a DP of 2 to 20, preferably 2 to 10. More preferably, these oligosaccharides do not contain substantial amounts (less than 5 weight %) of saccharides outside these DP ranges, and they are soluble. These oligosaccharides may comprise fructo-oligosaccharides (FOS), trans-galacto-oligosaccharides (TOS), xylo-oligosaccharides (XOS), soy oligosaccharides, and the like. Optionally, also higher molecular weight compounds such as inulin, soy polysaccharides, acacia polysaccharides (acacia fibre or arabic gum), cellulose, resistant starch and the like may be incorporated in the composition according to the invention. The amount of insoluble fibre such as cellulose is preferably lower than 20 weight % of the dietary fibre fraction of the composition according to the invention, and/or below 0.6 g/100 ml. The amount of thickening polysaccharides such as carrageenans, xanthans, pectins, galactomannans and other high molecular weight (DP>50) indigestible polysaccharides is preferably low, i.e. less than 20% of the weight of the fibre fraction, or less than 1 g/100 ml. Instead, hydrolysed polysaccharides such as hydrolysed pectins and galactomannans can advantageously be included.

A preferred fibre component is an indigestible oligosaccharide with a chain length (DP) of 2 to 10, for example Fibersol® (resistant oligoglucose), in particular hydrogenated Fibersol®, or a mixture of oligosaccharides having a DP of 2 to 10, such as fructo-oligosaccharides or galacto-oligosaccharides, which may also contain a small amount of higher saccharides (e.g. with a DP of 11 to 20). Such oligosaccharides preferably comprise 50 weight % to 90 weight % of the fibre fraction, or 0.5 g/100 ml to 3 g/100 ml of the composition according to the invention. Other suitable fibre components include saccharides that have only partial digestibility.

In a particular embodiment, the composition according to the invention comprises one or more of fructo-oligosaccharides, inulin, acacia polysaccharides, soy polysaccharides, cellulose and resistant starch.

In another embodiment of the present invention, the composition according to the invention may comprise a mixture of neutral and acid oligosaccharides as disclosed in WO 2005/039597 (N.V. Nutricia), which is incorporated herein by reference in its entirety. More in particular, the acid oligosaccharide has a degree of polymerisation (DP) between 1 and 5000, preferably between 1 and 1000, more preferably between 2 and 250, even more preferably between 2 and 50, most preferably between 2 and 10. If a mixture of acid oligosaccharides with different degrees of polymerisation is used, the average DP of the acid oligosaccharide mixture is preferably between 2 and 1000, more preferably between 3 and 250, even more preferably between 3 and 50. The acid oligosaccharide may be a homogeneous or heterogeneous carbohydrate. The acid oligosaccharides may be prepared from pectin, pectate, alginate, chondroitine, hyaluronic acids, heparine, heparane, bacterial carbohydrates, sialoglycans, fucoidan, fucooligosaccharides or carrageenan, and are preferably prepared from pectin or alginate. The acid oligosaccharides may be prepared by the methods described in WO 01/60378, which is hereby incorporated by reference. The acid oligosaccharide is preferably prepared from high methoxylated pectin, which is characterised by a degree of methoxylation above 50%. As used herein, “degree of methoxylation” (also referred to as DE or “degree of esterification”) is intended to mean the extent to which free carboxylic acid groups contained in the polygalacturonic acid chain have been esterified (e.g. by methylation). The acid oligosaccharides are preferably characterised by a degree of methoxylation above 20%, preferably above 50% even more preferably above 70%. Preferably the acid oligosaccharides have a degree of methylation above 20%, preferably above 50% even more preferably above 70%. The acid oligosaccharide is preferably administered in an amount of between 10 mg and 100 gram per day, preferably between 100 mg and 50 grams per day, even more between 0.5 and 20 gram per day.

The term neutral oligosaccharides as used in the present invention refers to saccharides which have a degree of polymerisation of monose units exceeding 2, more preferably exceeding 3, even more preferably exceeding 4, most preferably exceeding 10, which are not or only partially digested in the intestine by the action of acids or digestive enzymes present in the human upper digestive tract (small intestine and stomach) but which are fermented by the human intestinal flora and preferably lack acidic groups. The neutral oligosaccharide is structurally (chemically) different from the acid oligosaccharide. The term neutral oligosaccharides as used in the present invention preferably refers to saccharides which have a degree of polymerisation of the oligosaccharide below 60 monose units, preferably below 40, even more preferably below 20, most preferably below 10. The term monose units refer to units having a closed ring structure, preferably hexose, e.g. the pyranose or furanose forms. The neutral oligosaccharide preferably comprises at least 90%, more preferably at least 95% monose units selected from the group consisting of mannose, arabinose, fructose, fucose, rhamnose, galactose, β-D-galactopyranose, ribose, glucose, xylose and derivatives thereof, calculated on the total number of monose units contained therein. Suitable neural oligosaccharides are preferably fermented by the gut flora. Preferably the oligosaccharide is selected from the group consisting of: cellobiose (4-O-β-D-glucopyranosyl-D-glucose), cellodextrins ((4-O-β-D-glucopyranosyl)ₙ-D-glucose), B-cyclodextrins (Cyclic molecules of α-1-4-linked D-glucose; α-cyclodextrin-hexamer, β-cyclodextrin-heptamer and γ-cyclodextrin-octamer), indigestible dextrin, gentiooligosaccharides (mixture of β-1-6 linked glucose residues, some 1-4 linkages), glucooligosaccharides (mixture of α-D-glucose), isomaltooligosaccharides (linear α-1-linked glucose residues with some 1-4 linkages), isomaltose (6-O-α-D-glucopyranosyl-D-glucose); isomaltriose (6-O-α-D-glucopyranosyl-(1-6)-α-D-glucopyranosyl-D-glucose), panose (6-O-α-D-glucopyranosyl-(1-6)-α-D-glucopyranosyl-(1-4)-D-glucose), leucrose (5-O-α-D-glucopyranosyl-D-fructopyranoside), palatinose or isomaltulose (6-O-α-D-glucopyranosyl-D-fructose), theanderose (O-α-D-glucopyranosyl-(1-6)-O-α-D-glucopyranosyl-(1-2)-B-D-fructofuranoside), D-agatose, D-lyxo-hexylose, lactosucrose (O-β-D-galactopyranosyl-(1-4)-O-α-D-glucopyranosyl-(1-2)-β-D-fructofuranoside), α-galactooligosaccharides including raffinose, stachyose and other soy oligosaccharides (O-α-D-galactopyranosyl-(1-6)-α-D-glucopyranosyl-β-D-fructofuranoside), β-galactooligosaccharides or transgalacto-oligosaccharides (β-D-galactopyranosyl-(1-6)-[β-D-glucopyranosyl]ₙ-(1-4)α-D glucose), lactulose (4-O-β-D-galactopyranosyl-D-fructose), 4′-galatosyllactose (O-D-galactopyranosyl-(1-4)-O-β-D-glucopyranosyl-(1-4)-D-glucopyranose), synthetic galactooligosaccharide (neogalactobiose, isogalactobiose, galsucrose, isolactosel, II and III), fructans—Levan-type (β-D-(2→6)-fructofuranosyl)ₙ α-D-glucopyranoside), fructans—Inulin-type (β-D-((2→1)-fructofuranosyl)ₙ α-D-glucopyranoside), 1 f-β-fructofuranosylnystose (β-D-((2→1)-fructofuranosyl)ₙ B-D-fructofuranoside), xylooligosaccharides (B-D-((1→4)-xylose)ₙ, lafinose, lactosucrose and arabinooligosaccharides.

According to a further preferred embodiment the neutral oligosaccharide is selected from the group consisting of fructans, fructooligosaccharides, indigestible dextrins galactooligosaccharides (including transgalactooligosaccharides), xylooligosaccharides, arabinooligosaccharides, glucooligosaccharides, mannooligosaccharides, fucooligo-saccharides and mixtures thereof. Most preferably, the neutral oligosaccharide is selected from the group consisting of fructooligosacchararides, galactooligosaccharides and transgalactooligosaccharides.

Suitable oligosaccharides and their production methods are further described in Laere K. J. M. (Laere, K. J. M., Degradation of structurally different non-digestible oligosaccharides by intestinal bacteria: glycosylhydrolases of Bi. adolescentis. PhD-thesis (2000), Wageningen Agricultural University, Wageningen, The Netherlands), the entire content of which is hereby incorporated by reference. Transgalactooligosaccharides (TOS) are for example sold under the trademark Vivinal™ (Borculo Domo Ingredients, Netherlands). Indigestible dextrin, which may be produced by pyrolysis of corn starch, comprises α(1→4) and α(1→6) glucosidic bonds, as are present in the native starch, and contains 1→2 and 1→3 linkages and levoglucosan. Due to these structural characteristics, indigestible dextrin contains well-developed, branched particles that are partially hydrolysed by human digestive enzymes. Numerous other commercial sources of indigestible oligosaccharides are readily available and known to skilled person. For example, transgalactooligosaccharide is available from Yakult Honsha Co., Tokyo, Japan. Soybean oligosaccharide is available from Calpis Corporation distributed by Ajinomoto U.S.A. Inc., Teaneck, N.J.

In a further preferred embodiment the composition according to the invention comprises an acid oligosaccharide with a DP between 2 and 250, prepared from pectin, alginate, and mixtures thereof; and a neutral oligosaccharide, selected from the group of fructans, fructooligosaccharides, indigestible dextrins, galactooligosaccharides including transgalactooligosaccharides, xylooligosaccharides, arabinooligosaccharides, glucooligosaccharides, mannooligosaccharides, fucooligosaccharides, and mixtures thereof.

In a further preferred embodiment the composition according to the invention comprises two chemically distinct neutral oligosaccharides. It was found that the administration of acid oligosaccharides combined with two chemically distinct neutral oligosaccharides provides an optimal synergistic immune stimulatory effect. Preferably the composition according to the invention comprises:
an acid oligosaccharides as defined above;
a galactose-based neutral oligosaccharide (of which more than 50% of the monose units are galactose units), preferably selected from the group consisting of galactooligosaccharide and transgalactooligosaccharide; and
a fructose and/or glucose based neutral oligosaccharide (of which more than 50% of the monose units are fructose and/or glucose, preferably fructose units), preferably inulin, fructan and/or fructooligosaccharide, most preferably long chain fructooligosaccharide (with an average DP of 10 to 60).

The mixture of acid- and neutral oligosaccharides is preferably administered in an amount of between 10 mg and 100 gram per day, preferably between 100 mg and 25 grams per day, even more preferably between 0.5 and 20 gram per day.

### Viscosity and Osmolarity

In the context of this invention, the viscosity is measured in a rotational rheometer using a cone-plate geometry at 20° C. at a shear rate of 100 s⁻¹.

In one embodiment of the present invention, the viscosity of the liquid enteral nutritional composition is less than about 200 mPa·s, preferably less than 150 mPa·s, preferably less than 120 mPa·s. Other preferred embodiments show a viscosity of less than or equal to 80 mPas·s, preferably less than 70 mPa·s, more preferably less than 50 mPa·s, still more preferably less than 40 mPa·s, most preferably equal to about 20 mPa·s. or preferably between 20 and 45 mPa·s. The latter is ideal for orally administering the liquid enteral nutritional composition according to the invention because a person may easily consume a serving having a low viscosity such as that displayed by the present invention. This is also ideal for unit dosages that are tube fed.

In one embodiment of the present invention, the osmolarity of the composition is preferably lower than 900 mOsm/l, more preferably lower than 800 mOsm/l, most preferable lower than 700 mOsm/l.

In one embodiment of the present invention, the density of the composition ranges between 0.90 g/ml and 1.20 g/ml, preferably between 1.05 g/ml and 1.20 g/ml, especially between 1.10 g/ml and 1.18 g/ml.

### Dosage Unit

The liquid enteral nutritional composition according to the invention may have the form of a complete food, i.e. it can meet all nutritional needs of the user. As such, it preferably contains 1200 to 2500 kcal per daily dosage. The daily dosage amounts are given with respect to a daily energy supply of 2000 kcal to a healthy adult having a body weight of 70 kg. For persons of different condition and different body weight, the levels should be adapted accordingly. It is understood that the average daily energy intake preferably is about 2000 kcal. The complete food can be in the form of multiple dosage units, e.g. from 4 (250 ml/unit), 8 (125 ml/unit), 10 (100 ml/unit) to 20 (50 ml/unit) per day for an energy supply of 2000 kcal/day using a liquid enteral nutritional composition according to the invention of 2.0 kcal/ml.

The liquid enteral nutritional composition can also be a food supplement, for example to be used in addition to a non-medical food. Preferably as a supplement, the liquid enteral nutritional composition contains per daily dosage less than 1500 kcal, in particular as a supplement, the liquid enteral nutritional composition contains 400 to 1000 kcal per daily dose. The food supplement can be in the form of multiple dosage units, e.g. from 2 (250 ml/unit), 4 (125 ml/unit) to 10 (50 ml/unit) per day for an energy supply of 1000 kcal/day using a liquid enteral nutritional composition according to the invention of 2.0 kcal/ml.

In one embodiment of the present invention, a unit dosage comprises any amount of the liquid enteral nutritional composition according to the invention between 10 ml and 250 ml, the end values of this range included, preferably any amount between 25 ml and 200 ml, the end values of this range included, more preferably any amount between 50 ml and 150 ml, the end values of this range included, most preferably about 125 ml. For example, a person receiving 50 ml unit dosages can be given 10 unit dosages per day to provide nutritional support using a liquid enteral nutritional composition according to the invention of 2.0 kcal/ml. Alternatively a person receiving 125 ml unit dosages can be given 4 or 5 or 6 or 7 or 8 unit dosages per day to provide nutritional support using a liquid enteral nutritional composition according to the invention of 2.0 kcal/ml. Such small dosage units are preferred because of better compliance.

In one embodiment of the present invention, the composition is provided in a ready to use liquid form and does not require reconstitution or mixing prior to use. The composition according to the invention can be tube fed or administered orally. For example, the composition according to the invention can be provided in a can, on spike, and hang bag. However, a composition may be provided to a person in need thereof in powder form, suitable for reconstitution using an aqueous solution or water such that the composition according to the invention is produced. Thus in one embodiment of the present invention, the present composition is in the form of a powder, accompanied with instructions to dissolve or reconstitute in an aqueous composition or water to arrive at the liquid nutritional enteral composition according to the present invention. In one embodiment of the present invention, the present liquid nutritional enteral composition may thus be obtained by dissolving or reconstituting a powder, preferably in an aqueous composition, in particular water.

In one embodiment of the present invention, the composition according to the invention is packaged. The packaging may have any suitable form, for example a block-shaped carton, e.g. to be emptied with a straw, a carton or plastic beaker with removable cover, a small-sized bottle for example for the 80 ml to 200 ml range, and small cups for example for the 10 ml to 30 ml range. Another suitable packaging mode is inclusion of small volumes of liquid (e.g. 10 ml to 20 ml) in edible solid or semi-solid hulls or capsules, for example gelatine-like coverings and the like. Another suitable packaging mode is a powder in a container, e.g. a sachet, preferably with instructions to dissolve or reconstitute in an aqueous composition or water.

### Embodiments According to the Invention

In the following, a number of novel compositions are presented, comprising an intact protein having a low specific volume in solution. Said specific embodiments are the subject of co-pending applications PCT/NL2007/050626 and PCT/NL2008/050141, the content of which is incorporated herein by reference.

### 1. Micellar Casein and Caseinate

In a first embodiment, the liquid enteral nutritional composition comprises micellar casein and caseinate.

Micellar casein, also named native micellar casein, is a high quality milk protein and naturally occurring in milk in a concentration of about 2.6 g/100 ml (Dairy Science and Technology, Walstra et al., CRC Press, 2006). It is concentrated by a process that does not, or does not substantially denature the casein proteins and it is marketed as Micellar Casein Isolate (MCI). Fresh skim milk is subjected to a filtration process, in much the same process used to concentrate whey protein, to produce a pure, substantially undenaturated milk protein with its native structure. The resulting material contains more than 95 weight % micellar casein, the rest mainly being whey protein and other non-protein nitrogen and other constituents, such as lactose. It has an intrinsic low viscosity and a liquid composition comprising said MCI is therefore easy to drink.

In contrast, casein, as it is used in the context of this invention refers to the curd form of casein, having lost its native micellar structure.

Within the context of this invention, it is understood that micellar casein may also be provided by other milk protein sources, such as, for instance, sources with essentially preserve the natural 80:20 ratio of casein to whey, such as Milk Protein Concentrate (MPC), which is a powder product usually prepared by ultrafiltration with an average protein content of about 80 weight %, Milk Protein Isolate (MPI), a powder product usually prepared by precipitation with an average protein content of more than 85 weight %, and skimmed concentrated milk.

Although the composition of the present embodiment should not contain large amounts of proteins other than micellar casein and caseinate, the composition of the present embodiment may comprise up to about 30 weight %, in particular up to about 15 weight % of whey protein based on total protein without substantially affecting the viscosity and shelf-stability, even after pasteurisation and/or sterilisation.

Surprisingly, using a mixture of micellar casein and caseinate, did not increase the viscosity of the final composition as much as could be expected by the substitution of an amount of micellar casein by the same amount of caseinate, and consequently a composition is obtained after heat-treatment with still a low viscosity, which is still very easy to drink or to administer by tubing. By heat-treatment is meant any common treatment known to the skilled person to pasteurise or sterilise the composition of the present invention, in the manufacture of a nutritional composition.

According to one embodiment of the present invention, a liquid nutritional composition is provided comprising from about 4 g/100 ml to about 20 g/100 ml, preferably from about 6 g/100 ml to about 16 g/100 ml of protein, said protein comprising micellar casein and caseinate. Specific amounts are for instance about 7.5 g/100 ml, 8.0 g/100 ml, 9.6 g/100 ml and 12.5 g/100 ml.

According to one embodiment of the present invention, the combined amount of micellar casein and caseinate in the liquid nutritional composition according to the invention is at least 85 weight %, more preferably at least 90 weight %, more preferably at least 95 weight % of the total protein present in the liquid nutritional composition.

As aforementioned, the composition in this embodiment of the invention preferably does not contain large amounts of proteins other than micellar casein and caseinate. In a further embodiment of the present invention, the composition may comprise up to about 15 weight % of whey protein, preferably less than or equal to 10 weight % of whey protein, more preferably, less than or equal to 5 weight % of whey protein of the total protein present in the liquid nutritional composition.

In one embodiment of the present invention, Na-caseinate, Mg-caseinate, K-caseinate or any mixture thereof or combinations thereof such as Na/K-caseinate and Na/Mg caseinate are used as the source of caseinate. Preferably, Ca-caseinate, or a caseinate comprising Ca is not used, as the micellar casein already contains a sufficient amount of calcium, and the formation of further calcium crystals and other Ca-containing sediments preferably is avoided.

According to one embodiment of the present invention, the weight ratio of micellar casein to caseinate ranges from 90:10 to 50:50. Preferably, the weight ratio of micellar casein to caseinate is equal to 60:40.

The aforementioned liquid enteral nutritional composition according to the invention may be prepared by first preparing the liquid protein composition. This may be done by sequentially or simultaneously dissolving micellar casein in powder form and caseinate in powder form in water. It is also possible to use micellar casein in a wet form, directly prepared from milk. It may even be advantageous to prepare the micellar casein as a part of a continuous process to prepare the composition according to the invention. The latter may be done in the same production facility to prepare the composition according to the invention.

Furthermore, if the liquid enteral nutritional composition is to contain further components, a nutritional product may be prepared by subsequently adding the digestible carbohydrates to the protein composition, followed by adding the water-soluble vitamins and other components in one or two stages, mixing, adjusting the resulting composition to the desired viscosity, adding the fat, including fat-soluble vitamins, homogenizing, subjecting the resulting solution to a heat-treatment (pasteurization, sterilisation) and packaging the resulting product. In this respect, it is noted that the acidity of the composition is very important during the heat-treatment. The pH should be between about 6.0 and 7.2 for the pasteurisation and sterilisation. Typical pasteurisation time/temperature-combinations range between 15 sec at 80° C. to 2 minutes at 135° C. A typical sterilisation time/temperature-combination is 4 minutes at 124° C.

The aforementioned composition according to the invention is designed to either supplement a person's diet or to provide complete nutritional support. Hence, the composition according to the invention may further comprise at least fat and/or digestible carbohydrate and/or a source of vitamins and minerals and/or a source of prebiotics. Preferably, the composition according the invention is a nutritionally complete composition.

### 2. Globular Protein

In an embodiment of the present invention, a liquid enteral nutritional composition is provided comprising an amount of heat-treated non-hydrolysed globular protein, wherein the non-hydrolysed globular protein, in particular whey protein, is obtained by a heat-treatment, comprising the consecutive steps of:
a) adjusting the pH of an aqueous composition comprising non-hydrolysed globular proteins to a value of between about 2 and 8;
b) converting the composition comprising non-hydrolysed globular proteins obtained in step a) into an aerosol;
c) subjecting the aerosol obtained in step b) to a temperature of 100 to 190° C. during a time of about 10 to 300 milliseconds;
d) flash-cooling the heat-treated aerosol obtained in step c) to a temperature below 85° C. to obtain an aqueous solution comprising heat-treated globular proteins.

In one embodiment the globular protein comprises a protein selected from the group consisting of whey protein, pea protein, soy protein, and any mixture thereof. In particular, said globular protein comprises a whey protein. In one embodiment the whey protein is selected from the group consisting of β-lactoglobulin, α-lactalbumin, serum albumin, or any mixture thereof. In one embodiment the whey protein comprises whey protein concentrate (WPC), whey protein isolate (WPI), or any mixture thereof.

It is noted that the aqueous composition comprising heat-treated non-hydrolysed globular proteins may contain, next to the heat-treated non-hydrolysed globular proteins, in particular whey protein, any other nutritional ingredients, such as other proteins, amino acids, fat, digestible carbohydrates, fibers, minerals, vitamins, and the like, and that these ingredients may be present when subjecting the aqueous composition to the method according to the invention, in particular step b).

In one embodiment, the pH of the aqueous composition of non-hydrolysed globular proteins in step a) is about 2 to 5. More preferably, the pH of the aqueous composition of non-hydrolysed globular proteins in step a) is about 4.

In yet another embodiment, the pH of the aqueous composition of non-hydrolysed globular proteins in step a) is about 6 to 8. More preferably, the pH of the aqueous composition of non-hydrolysed globular proteins in step a) is about 7.

In one embodiment, the aerosol obtained in step b) is subjected to a temperature of 110 to 180° C., during a time of about 20 to 200 milliseconds, more preferably 40 to 150 milliseconds, more preferably 80 to 120 milliseconds. In another embodiment, the aerosol obtained in step b) is subjected to a temperature of 110° C., during a time of about 20 to 200 milliseconds, more preferably 40 to 150 milliseconds, more preferably 80 to 120 milliseconds. In yet another embodiment, the aerosol obtained in step b) is subjected to a temperature of 170° C., during a time of about 20 to 200 milliseconds, more preferably 40 to 150 milliseconds, more preferably 80 to 120 milliseconds.

In one embodiment, in step a) the pH of an aqueous composition of non-hydrolysed whey proteins is adjusted to a value of about 4 (acid whey protein solution), and the aerosol obtained in step b) is subjected to a temperature of 110° C., during a time of about 20 to 200 milliseconds, more preferably 40 to 150 milliseconds, more preferably 80 to 120 milliseconds.

In another embodiment, in step a) the pH of an aqueous composition of non-hydrolysed whey proteins is adjusted to a value of about 7 (neutral whey protein solution), and the aerosol obtained in step b) is subjected to a temperature of 170° C., during a time of about 20 to 200 milliseconds, more preferably 40 to 150 milliseconds, more preferably 80 to 120 milliseconds.

In one embodiment, in step c), the conversion of the composition of non-hydrolysed globular proteins obtained in step a) into an aerosol is done using a spray nozzle, as described below in detail.

In one embodiment, step d) is performed by transporting the aerosol into a vacuum chamber (flash-cooling) to remove an amount of water by evaporation, equivalent to the amount of steam used and the product is cooled by indirect cooling to a temperature of less than about 85° C., preferably less than about 60° C. This method allows fast cooling and quick removal of volatiles (i.e. steam). The cooling preferably takes place nearly instantaneously, i.e. in a time window preferably of milliseconds.

It is without saying that any of the aforementioned preferred values (pH, temperature and time) and ranges thereof for each of the steps a), b), c) and d) may be combined in an intelligent manner, without departing from the scope of the invention.

The apparatus to carry out this particular embodiment may be selected by the skilled person based on the steps described above. Basically, the apparatus to carry out the invention comprises a nozzle for atomizing the composition (step b), a chamber to heat the aerosol (step c), and a chamber to cool the heated aerosol (step d). Preferably, the heating is done by mixing the aerosol with steam of a certain temperature (and at a certain steam pressure). When using steam, the apparatus may comprise a nozzle and a mixing chamber. A mixing chamber generally comprises one or more inflow openings for steam flows and for product flows, in which a product flow may optionally be premixed with a part of the steam. It may be preferable to select the mixing chamber such that only one product flow is atomized with one steam flow, since this simplifies the cleaning of the mixing chamber after use.

A schematic representation of a suitable nozzle for atomization according to the invention is shown in EP 1351587, FIG. 1, in which a nozzle with mixing chamber is shown. Said FIG. 1 is incorporated herein by reference. It turns out that a nozzle with mixing chamber can be very effectively used for the heat treatment of the product. A suitable mixing chamber is generally characterized in that steam and atomized product to be treated are mixed, while the volume throughput of the steam will be much greater than that of the atomized product to be treated and the residence time of the atomized product is sufficiently to obtain the desired heat-treated globular protein. The volume ratio between the steam flow and the product flow may range between, for instance, about 20:1 and 150:1. It is important that the pressure in the mixing chamber is higher than in the space to which the atomized product is passed.

The form and size of the inflow openings for the steam flow (1) and the flow of the product in liquid form (2) in the mixing chamber and their mutual position are selected such that intensive mixing takes place between product and steam. It is noted that the inflow openings can be placed such that the steam flow and the product flow enter the mixing chamber in substantially parallel direction. This may take place in both a horizontally, vertically and diagonally manner. However, it is also possible that the steam flow and the product flow enter the mixing chamber at different angles, for instance a vertical steam flow and a horizontal product flow. The inflow openings are further arranged such that the product is atomized in small droplets, which after a short residence time in the mixing chamber (4) leave the mixing chamber through an outflow opening (5), for instance to a cooling chamber (6). The inflow opening(s) for the steam flow preferably contain a steam distribution plate (3). By changing the dimensions of the mixing chamber and/or the outflow opening(s) in the manner known to those skilled in the art, the average residence time and particle size of the atomized droplets can be varied. To set a suitable residence time in the mixing chamber is a simple matter of optimization for the skilled person and depends at least on the temperature and pressure in the mixing chamber.

The mixing is preferably carried out by contacting the atomized product flow and the steam flow close to the inflow opening of the product in the mixing chamber and bringing the steam at high speed around the atomized product. In a preferred embodiment, such a mixing takes place by bringing the steam near the product concentrically around the inflow opening of atomized product in the mixing chamber. The product flow to steam flow ratio can be varied in a ratio of 1.6 to 10 kg product in liquid form per kg steam. Very good results are obtained at a wet product flow to steam flow ratio of 2.4 to 8 kg product in liquid form per kg steam.

In principle, any type of mixing chamber is suitable in which steam and product can be mixed and atomized. Very suitable for mixing and atomizing a product-steam mixture according to the invention is a nozzle such as “two-fluid” type nozzle, an example of which is described in EP 0438783, FIG. 1, which is incorporated herein by reference. This nozzle contains a small chamber at the end of a product line in which steam and product are combined. To increase the capacity, more nozzles can be used in a parallel arrangement.

The temperature of the supplied saturated or superheated steam in the method according to the invention is preferably in the range of 100 and 190° C., more preferably between 100 and 180° C., still more preferably between 100 and 170° C. In general, the temperature in the mixing chamber will be maintained at the desired level through the steam, although it is also possible that the mixing chamber itself is heated by another heat source.

Good results are obtained when introducing the steam into the mixing chamber at a steam pressure of 1.5 to 10 bar, and in particular at a steam pressure of 1.8 to 8.2 bar, in mixing chambers about 1 to 20 cm in length. This pressure is preferably measured just before the steam is introduced into the mixing chamber via a spray nozzle.

Preferably, the particle size (aggregate size) of the flash-cooled aerosol (obtained in step d) is less than about 30 μm, more preferably less than about 10 μm, still more preferably less than 5 μm, and most preferably less than 1 μm. At particle diameters above 30 μm, the nutritional composition may start to taste sandy, which is not favoured.

Depending on the temperature/time combination, the method according to the invention may not provide sufficient pasteurization or sterilization. For instance, 100 milliseconds at 110° C. does not provide sufficient microbial sterility for a neutral product. However, 100 milliseconds at 170° C. would provide sufficient sterility.

In one embodiment, the product obtained from step d) is further pasteurized using conventional equipment such as a plate or tubular heat exchanger, scraped surface heat exchanger or a retort to give the final product. Most excellent results are obtained when using a plate heat exchanger. The invention therefore also relates to the above described method according to the invention, comprising steps a), b), c) and d), subsequently comprising a pasteurization step using a plate heat exchanger. The plate heat exchanger is preferably operated at 92° C. for 30 seconds.

In another embodiment, a sterile product is obtained from step d) or from the above mentioned subsequent pasteurization step, which can be filled aseptically in aseptic containers in a further process step.

In another embodiment of the composition according to the invention, the amount of non-hydrolysed globular proteins is about 9, 10, 11, 12, 13, 14, 15, 16, 17, 18, 19 or 20 gram per 100 ml of the composition, or any value in between the aforementioned values.

In one embodiment of the composition according to the invention, the pH of the liquid enteral nutritional composition is about 2 to about 8, preferably about 4 to about 7. In another embodiment of the composition according to the invention, the pH is about 2, 3, 4, 5, 6, 7, or 8 or any value in between the aforementioned values.

In one embodiment, the liquid enteral nutritional composition has an energy density of at least 1.5 kcal/ml, preferably at least 2.0 kcal/ml. In a specific embodiment of the composition according to the invention, the composition is acidic (yoghurt-like or juice-like) with a pH of about 4. Acidification may be achieved by any method known to the skilled person, such as the addition of an acid (such as, for instance lactic acid, citric acid, phosphoric acid) or through fermentation. The thus obtained composition has a pleasant mild acidic taste which can be flavoured perfectly with a fruity flavour.

In a further specific embodiment of the invention, the composition has a neutral pH (i.e. a pH of about 7). The thus obtained composition has a pleasant taste which may optionally be flavoured with e.g. vanilla, chocolate, caramel, banana, strawberry.

In one embodiment the amount of non-hydrolysed globular protein, preferably whey protein, in the liquid nutritional composition according to the invention is at least 85 weight %, more preferably at least 90 weight %, more preferably at least 95 weight % of the total protein present in the liquid nutritional composition.

In a further embodiment of the present invention, the composition further comprises a non-globular protein. In one embodiment the non globular protein is selected from the group consisting of casein, caseinate, micellar casein isolate and the like, and any mixture thereof. In one embodiment the composition comprises up to about 40 weight % of a non-globular protein, such as casein, caseinate, micellar casein isolate and the like, and any mixture thereof, preferably less than or equal to 20 weight %, more preferably less than or equal to 10 weight % of the total protein present in the liquid nutritional composition.

In one embodiment of the present invention, the composition may comprise a free amino acid, or a mixture of free amino acids, up to 5 g/100 ml, more preferably less than 2 g/100 ml, more preferably less than 1 g/100 ml, most preferably less than 0.5 g/100 ml

### Effectivity

The present invention also concerns a method of providing nutrition to a person in need thereof, comprising the steps of administering to said person the nutritional composition according to the present invention. Said person may be an elderly person, a person that is in a disease state, a person that is recovering from a disease state, or a person that is malnourished.

Said person may also be a healthy person, such as a sportsman or a sportswoman or an active elderly.

The invention will now be further elucidated by several examples, without being limited thereby.

### EXAMPLES

### A1-A4. Compositions Based on Micellar Casein and Caseinate

The following compositions according to the invention have been prepared (Table 1). The compositions are produced in a manner known per se, e.g. by mixing the ingredients, without difficulties, are shelf-stable, have desirable organoleptic properties, have a very high nutrient density and are effective for a person in need thereof.

TABLE 1

| | Amount per 100 ml of product | | | | | |
|---|---|---|---|---|---|---|
| Component | A1 | A2 | A3 | A4 | A7 | A8 |
| Energy (kcal/100 ml) | 200 | 240 | 280 | 340 | 160 | 160 |
| Protein (En %) | 25 | 16 | 11 | 9 | 40 | 40 |
| Protein (g) | 12.5 | 9.6 | 8.0 | 7.5 | 16 | 16 |
| MCI:Na-caseinate | 80:20 | 65:35 | 80:20 | 80:20 | 80:20 | 96:4 |
| (wt %/wt %) | | | | | | |
| Fat (En %) | 27 | 35 | 47 | 73 | 30 | 30 |
| Fat (g) mainly comprising | 6.0 | 9.3 | 14 | 28 | 5.3 | 5.3 |
| canola oil | | | | | | |
| Carbohydrates (En %) | 48 | 49 | 42 | 18 | 30 | 30 |
| Maltodextrose (DE 47) | 24.0 | 29.4 (A2′) | 29.5 | 16 | | |
| (Exp. A2′) in g | | | | | | |
| Mixture of lactose/ | | 29.4 (A2″) | | | 12 | 12 |
| sucrose/tre-halose/ | | | | | | |
| palatinose/malto- | | | | | | |
| dextrin (Exp. A2″) in g | | | | | | |
| Minerals | 16% of RDI | 16% of RDI | 16% of RDI | 16% of RDI | 16% of RDI | 16% of RDI |
| Vitamins | 16% of RDI | 16% of RDI | 16% of RDI | 16% of RDI | 16% of RDI | 16% of RDI |
| Viscosity (mPa · s | 75 | 70 | 75 | 75 | 75 | 75 |
| at 20° C. at 100 s⁻¹) | | | | | | |
| Density (g/ml) | 1.18 | 1.16 | 1.12 | 1.06 | n.d | n.d |
| Unit dosage (ml) | 125 | 125 | 125 | 125 | 125 | 125 |

n.d.: not determined

### A5-A6. Compositions Based on Heat-Treated Non-Hydrolysed Whey Proteins

A number of compositions were manufactured using the method as described below. These compositions are summarized in Table 2.

TABLE 2

Nutritional
composition

| Component | A5 | A6 |
|---|---|---|
| Energy value (kcal/ml) | 2.4 | 2.4 |
| Protein | WPI | WPI |
| (g/100 ml) | 16 | 16 |
| (En %) | 27 | 27 |
| Fat | | |
| (g/100 ml) | 8.5 | 8.5 |
| (En %) | 31 | 31 |
| Carbohydrates | | |
| (g/100 ml) | 25 | 25 |
| (En %) | 42 | 42 |
| Dietary fibre | 0 | 0 |
| Vitamins and Minerals | % of RDI | % of RDI |
| Final pH | 4.1 | 7.5 |
| Viscosity | 162 | 97 |

### Composition A5

### Acidic Whey Protein Composition (16 g/100 ml)

The protein (WPI) (Bipro®, Davisco), carbohydrates and minerals were dispersed in water and the solution was set to pH 4.1 using citric acid. The oil was blended into the product and the pre-emulsion was homogenised at 40° C. in a 2-stage homogenizer at a pressure of 550/50 bar. The product was then atomised into the spray-cooking chamber and instantly heated to 120° C. by mixing with steam and held at this temperature for approximately 50 msec. Subsequently, the product was flash-cooled to 50° C. and pumped into a holding tank. The final pH of the product was adjusted to pH 4.1 and the product was split in two batches. One batch was then UHT pasteurised at 92° C. for 30 sec and filled aseptically into aseptic 200 ml bottles. The product was liquid, with a viscosity of 75 mPa·s at a shear rate of 100 s⁻¹. The other batch was retorted (15 minutes at 92° C.). This product was liquid with a viscosity of 162 mPa·s at a shear rate of 100 s⁻¹. Both products had a smooth mouth feel. This is confirmed by the particle size distribution, which shows that the spray-cooking has little effect on the particle size. Moreover, the spray-cooking step appears to have stabilised the protein aggregates against further aggregation: the particle size after UHT and retort pasteurisation is nearly unchanged compared to the spray-cooked intermediate product. The average particle diameter as obtained from static light scattering (Malvern Mastersizer 2000), d[4,3], after homogenisation (a), spray-cooking (b) and UHT pasteurisation (c) or retorting (d) were 4.7 μm, 3.7 μm, 3.9 μm or 3.8, respectively. It was observed that the mineral levels had only small effects on the final product characteristics like particle size, viscosity and shelf life stability.

### Composition A6

### Neutral Whey Protein Composition (16 g/100 ml)

The whey protein isolate (WPI) (Bipro®, Davisco) and sucrose were dispersed in demineralised water and the solution was adjusted to pH 7.5 using a 10% KOH solution. Oil was blended into the product and the pre-emulsion was homogenised at 40° C. in a 2-stage homogenizer at a pressure of 550/50 bar. The product was then atomised into the spray-cooking chamber and instantly heated to 170° C. by mixing with steam and held at this temperature for approximately 100 ms. Subsequently, the product was flash-cooled to 55° C. and aseptically filled into aseptic 200 ml bottles. The product was liquid, with a viscosity of 97 mPa·s at a shear rate of 100 s⁻¹. The product had a smooth mouth feel. This is confirmed by the particle size distribution which shows that the spray-cooking has little effect on the particle size. If anything, the particles become smaller during the process. The average particle diameter as obtained from static light scattering (Malvern Mastersizer 2000), d[4,3], after homogenisation (a), spray-cooking (b) were 0.48 μm and 0.29 μm, respectively.

### Calculation of Specific Volume and Volume Fraction.

For a number of compositions according to the prior art (Comp. Exp.) and according to the invention (Exp. A1 to A6) specific volume and volume fraction were calculated and compared. All values are calculated for a 100 ml composition (Table 3). It is clear that specific volumes of compositions according to the invention are less than 3.30 ml/g, while prior art specific volumes of compositions are above 3.30 ml/g.

TABLE 3

| | | Enₙ | Cₚ | C_{c} | C_{f} | Others | Viscosity | | vₚ |
|---|---|---|---|---|---|---|---|---|---|
| Experiment | Commercial Name | (kcal/ml) | (g/100 ml) | (g/100 ml) | (g/100 ml) | (g/100 ml) | (mPa · s) | φₙ | (ml/g) |
| Comp. Exp. | RESOURCE 2.0 | 2.0 | 9 | 21.4 | 8.7 | fiber 2.5 g | 75 | 0.688 | 3.61 |
| | (Novartis) | | | | | | | | |
| Comp. Exp. | VHC 2.25 | 2.25 | 9 | 19.7 | 12 | no fiber | 80 | 0.692 | 3.90 |
| | (Nestle) | | | | | | | | |
| Comp. Exp. | FRESUBIN 2.0 | 2.0 | 10 | 22.5 | 7.8 | fiber 1.6 g | 56 | 0.669 | 3.30 |
| | (Fresenius) | | | | | | | | |
| Comp. Exp. | PRO-CAL SHOT | 3.34 | 6.7 | 13.4 | 28.2 | no fiber | 80 | 0.692 | 3.52 |
| | (VitaFlo) | | | | | | | | |
| Comp. Exp. | TwoCal HN | 2.0 | 8.4 | 21.6 | 8.9 | fiber 0.5 g | 130 | 0.713 | 4.36 |
| | (Abott) | | | | | | | | |

| | | | | | | | | |
|---|---|---|---|---|---|---|---|---|
| Exp. A1 | 2.4 | 9.6 | 29.3 | 9.3 | no fiber | 75 | 0.684 | 2.82 |
| Exp. A2 | 2.0 | 12.5 | 24.0 | 6.0 | no fiber | 70 | 0.688 | 2.95 |
| Exp. A3 | 2.8 | 8.0 | 29.5 | 14 | no fiber | 75 | 0.688 | 2.77 |
| Exp. A4 | 3.4 | 7.5 | 16 | 28 | no fiber | 75 | 0.688 | 2.75 |
| Exp. A5 | 2.4 | 16 | 25 | 8.5 | no fiber | 162 | 0.724 | 2.29 |
| Exp. A6 | 2.4 | 16 | 25 | 8.5 | no fiber | 97 | 0.702 | 2.15 |
| Exp A7 | 1.6 | 16 | 12 | 5.3 | no fiber | 75 | n.d. | 3.1 |
| Exp A8 | 1.6 | 16 | 12 | 5.3 | no fiber | 75 | n.d. | 3.1 |

### Relationship Between Energy Content of a Nutritional Composition (Enₙ) and the Protein Concentration (Cₚ), According to the Invention.

FIGS. 1a-k show the relationship between energy content of a nutritional composition (Enₙ) and the concentration of protein (Cₚ), according to the invention for different concentrations of fat (10, 20, 30, 35, 40, 50, 60, 70, 76, 80 and 90 En %). The commercially available prior art products have been indicated in the figures (bullets).

In FIG. 1d the bullet represents Comparative Example 3.

In FIG. 1e bullet A represents Comparative Example 5 and bullet B represents Comparative Example 1.

In FIG. 1f the bullet represents Comparative Example 2.

In FIG. 1i the bullet represents Comparative Example 4.

It should be understood that various changes and modifications to the presently preferred embodiments described herein will be apparent to those skilled in the art. Such changes and modifications may be made without departing from the spirit and scope of the invention and without diminishing its advantages. It is therefore intended that such changes and modifications are covered by the appended claims.

1.-15. (canceled)

16. A liquid enteral nutritional composition comprising an intact milk and/or plant protein having a specific volume in the composition of less than 3.30 ml/g.

17. The nutritional composition according to claim 16, having a specific volume in the composition of less than 3.25 ml/g.

18. The nutritional composition according to claim 17, having a specific volume in the composition of less than 3.20 ml/g.

19. The nutritional composition according to claim 16, which is sterilized or pasteurized.

20. The enteral nutritional composition according to claim 16, further comprising one or more of digestible carbohydrate, dietary fiber and fat.

21. The enteral nutritional composition according to claim 16, wherein the specific volume vₚ of the intact protein is calculated using the formulas (f1) and (f2)
φₙ=Cₚ·vₚ+C_{c}·v_{c}+C_{d}·v_{d}+C_{f}·v_{f} (f1)
ηₙ=η_{solvent{}1+1.25φₙ/[φₘₐₓ−φₙ)/φₘₐₓ]}² (f2)
wherein
the indices n, p, c, d and f stand for nutritional composition (n), intact protein (p), digestible carbohydrate (c), dietary fiber (d) and fat (f), respectively;
ηₙ is the measured viscosity in mPa·s;
ηₛₒₗᵥₑₙₜ=1 mPa·s
φₙ is the volume fraction;
φₘₐₓ=0.79
C is the absolute concentration of a material; and
v is the specific volume.

22. The enteral nutritional composition according to claim 16, comprising an intact protein, and optionally, one or more of digestible carbohydrate, dietary fiber and fat, wherein the relationship between the intact protein and any digestible carbohydrate, dietary fiber and fat and the volume fraction is described by formula (f6):
[Cₚ·v_{p′}+C_{c}·1.05+C_{d}·1.05+C_{f}·1.12]/φₙ>1 (f6)
wherein
the indices n, p, c, d and f stand for nutritional composition (n), intact protein (p), digestible carbohydrate (c), dietary fiber (d) and fat (f), respectively;
φₙ is the volume fraction;
C is the absolute concentration of a material;
v is the specific volume;
φₙ is defined as in formula (f1); and
v_{p′} is equal to 3.30.

23. The enteral nutritional composition according to claim 16, having a viscosity of less than 200 mPa·s.

24. The enteral nutritional composition according to claim 16, having a viscosity of less than 80 mPa·s.

25. The enteral nutritional composition according to claim 16, having a viscosity of less than 50 mPa·s.

26. The enteral nutritional composition according to claim 16, wherein the intact milk protein is selected from the group consisting of micellar casein and whey protein.

27. The enteral nutritional composition according to claim 16, comprising micellar casein and caseinate.

28. The enteral nutritional composition according to claim 27, comprising from about 4 g/100 ml to about 20 g/100 ml protein.

29. The enteral nutritional composition according to claim 27, comprising from about 6 g/100 ml to about 16 g/100 ml protein.

30. The enteral nutritional composition according to claim 27, wherein the combined amount of micellar casein and caseinate is at least 85 weight % of the total protein.

31. The enteral nutritional composition according to claim 17, further comprising whey protein.

32. The enteral nutritional composition according to claim 16, comprising heat-treated non-hydrolysed globular protein, wherein the non-hydrolysed globular protein is prepared by:
a) adjusting the pH of an aqueous composition comprising non-hydrolysed globular proteins to a value of between about 2 and 8;
b) converting the aqueous composition from a) into an aerosol;
c) subjecting the aerosol to a temperature of 100 to 190° C. for about 10 to 300 milliseconds; and
d) flash-cooling the heat-treated aerosol from c) to a temperature below 85° C.

33. The enteral nutritional composition according to claim 32, wherein the globular protein comprises a protein selected from the group consisting of whey protein, in particular whey protein concentrate (WPC), whey protein isolate (WPI), or any mixture thereof, pea protein, soy protein, and any mixture thereof.

34. A method of providing nutrition to a person in need thereof, comprising administering to the person the nutritional composition according to claim 16.

35. The method according to claim 34, wherein the person is of age 50 years or more, a person that is in a disease state, a person that is recovering from a disease state, a person that is malnourished, or a healthy person such as a sportsman or sportswoman or an active elderly.
